(19) United States
(12) Patent Application Publication
(10) Pub. No.: US 2023/0174395 A1
(43) Pub. Date: Jun. 8, 2023

(54) DISINFECTION SYSTEM USING PLASMA-DISCHARGED WATER, AND SPRAY NOZZLE FOR SPRAYING PLASMA-DISCHARGED WATER AS DROPLETS

(71) Applicant: KOREA INSTITUTE OF FUSION ENERGY, Daejeon (KR)

(72) Inventors: Yong Cheol HONG, Goyang-si (KR); Hee Jae LEE, Goyang-si (KR); Kang Il KIM, Jeonju-si (KR)

(73) Assignee: KOREA INSTITUTE OF FUSION ENERGY, Daejeon (KR)

(21) Appl. No.: 17/906,688

(22) PCT Filed: Jan. 29, 2021
(86) PCT No.: PCT/KR2021/001204

§ 371 Date: Sep. 19, 2022

(30) Foreign Application Priority Data
Mar. 26, 2020 (KR) 10-2020-0036967

(51) Int. Cl. C02F 1/46; A61L 2/22; A61L 2/26; A61L 9/22

(52) U.S. Cl. CPC C02F 1/4608; A61L 2/22; A61L 2/26; A61L 9/22; C02F 2303/04; A61L 2202/11; A61L 2202/15; A61L 2209/213; A61L 2209/134

(57) ABSTRACT

Disclosed are a disinfection system using plasma-discharged water, and a spray nozzle. The disinfection system using plasma-discharged water is characterized by comprising: a plasma-discharged water generating device; a to-be-treated water supply unit for supplying water to be treated to the plasma-discharged water generating device; a plasma-discharged water ejection unit connected to one side of the plasma-discharged water generating device; and a spray nozzle which is fluid-communicably connected to the plasma-discharged water generating device and supplied with plasma-discharged water from the plasma-discharged water ejection unit, and atomizes and sprays the supplied plasma-discharged water as droplets.

### FIELD OF THE INVENTION

The present disclosure relates to a disinfection system, and more specifically, relates to a disinfection system using treated water including a plasma-discharge treated water generation device for generating plasma-discharge treated water, and a spray nozzle for atomizing the plasma-discharge treated water into droplets and spraying the droplets.

### DESCRIPTION OF RELATED ART

In general, some of the bacteria or viruses that live widely in an environment in which humans live are pathogens that cause various diseases. In particular, some pathogens cause food poisoning through food, and some pathogens are transmitted through the air, thus causing many casualties in a short period of time.

In one example, norovirus is a causative agent of food poisoning transmitted through food. When the human being is infected therewith, the human undergoes vomiting, diarrhea and dehydration. Ebola virus is transmitted through direct contact with human body fluids, secretions, and blood. When the human being is infected with Ebola virus, she/he undergoes sudden fever, headache, and muscle pain, followed by general weakness, skin rash, and generalized bleeding. Yersinia pestis is transmitted to humans through fleas that parasitize rats as a host animal. When the human being is infected therewith, she/he undergoes symptoms such as muscle pain, headache or vomiting, diarrhea or coughing, and chest pain along with sudden fever.

Therefore, when an infected person is infected with a disease-causing bacteria or virus, an area where the infected person is located is quickly controlled to prevent the bacteria and virus from spreading to a surrounding area, and at the same time, the bacteria and virus remaining in the infected area must be quickly sterilized.

A conventional disinfection scheme for sterilizing bacteria and viruses includes spray disinfection and smoke screen disinfection. The spray disinfection refers to a scheme of spraying a mixture of chemicals with water. Although a disinfection area is small in this scheme, the scheme minimizes environmental pollution and the chemicals remain, thus achieving a high insecticidal effect. The smoke screen disinfection has a large disinfection area. Further, even in areas where air flow is blocked, such as in densely forested areas, insecticide particles may reach a deep position. However, the chemicals may not remain.

A disinfection device using the conventional disinfection scheme sprays the chemicals in an aerosol state or generates a smoke screen containing the chemicals and thus has cause high cost and environmental pollution.

Therefore, research on a disinfection scheme that may replace the conventional disinfection scheme using the chemicals is being actively conducted. A technology using atmospheric pressure plasma is emerging as a representative thereof.

Plasma discharge produce a variety of chemical effects on liquids in gaseous and liquid environments. That is, under the plasma discharge, various chemically active species such as radicals penetrate into the liquid, melt therein, and thus have chemical and bactericidal properties in the liquid.

As such, when plasma-discharge treated water having chemical sterilization properties under the plasma discharge in the gas and liquid environments is used for disinfection, a disinfection system without environmental pollution may be implemented.

Furthermore, when the plasma-discharge treated water may be sprayed in an aerosol state so that the plasma-discharge treated water stays in a suspended state for a longer time in the atmosphere. Thus, effective disinfection may be achieved.

### SUMMARY OF THE INVENTION

Therefore, a purpose of the present disclosure is to provide a disinfection system using plasma-discharge treated water that atomizes the plasma-discharge treated water and sprays the atomized plasma-discharge treated water in an aerosol form, thereby effectively conducting sterilization of bacteria and viruses.

Further, a purpose of the present disclosure is to provide a spray nozzle that may easily atomize the plasma-discharge treated water and spray the atomized plasma-discharge treated water in an aerosol form.

A first aspect of the present disclosure provides a disinfection system using plasma-discharge treated water, the system comprising: a plasma-discharge treated water generation device to generate the plasma-discharge treated water; a treatment target water supply for supplying treatment target water to the plasma-discharge treated water generation device; a plasma-discharge treated water discharger connected to one side of the plasma-discharge treated water generation device; and a spray nozzle connected to the plasma-discharge treated water generation device in a fluid communication manner, wherein the spray nozzle is constructed to: receive the plasma-discharge treated water from the plasma-discharge treated water discharger; and atomize the received plasma-discharge treated water into droplets and spray the droplets.

In one implementation of the system, the plasma-discharge treated water generation device includes: a chamber receiving therein the treatment target water; and in-water plasma discharge means received in an inner space of the chamber so as to generate plasma in the treatment target water.

In one implementation of the system, the in-water plasma discharge means includes: a metal tip receiving an electrical power; and a dielectric tube surrounding the metal tip and protruding by a predefined length beyond a distal end of the metal tip.

In one implementation of the system, the in-water plasma discharge means includes: a metal tip receiving an electrical power; a dielectric tube surrounding the metal tip and protruding by a predefined length beyond a distal end of the metal tip; and a gas supply channel extending through the metal tip in a longitudinal direction of the tip.

In one implementation of the system, the in-water plasma discharge means includes: a high voltage electrode to which a high voltage is applied; an inner dielectric tube surrounding the high voltage electrode; and an outer dielectric tube constructed to accommodate therein the inner dielectric tube so that an inner face of the outer dielectric tube is spaced, by a predefined spacing, from an outer face of the inner dielectric tube, wherein a plurality of through-holes are defined in a wall of the outer dielectric tube and are arranged in a length direction of the outer dielectric tube, wherein a source gas is injected through the plurality of through-holes into the outer dielectric tube, wherein the in-water plasma discharge means generates plasma using the source gas such that the generated plasma is directed from the outer dielectric tube toward the treatment target water.

In one implementation of the system, the plasma-discharge treated water generation device includes: a chamber having: first and second inner spaces defined therein and adjacent to each other and connected to each other in a fluid communicate manner, wherein the treatment target water is received in the first space, and the plasma-discharge treated water discharger is connected to the second space; and a gas outlet connected to the first inner space in a fluid communicate manner; and in-water plasma discharge means received in the second space so as to generate plasma in the treatment target water flowing from the first space toward the plasma-discharge treated water discharger, wherein the in-water plasma discharge means includes: a high voltage electrode to which a high voltage is applied; an inner dielectric tube surrounding the high voltage electrode; and an outer dielectric tube constructed to accommodate therein the inner dielectric tube so that an inner face of the outer dielectric tube is spaced, by a predefined spacing, from an outer face of the inner dielectric tube, wherein a plurality of through-holes are defined in a wall of the outer dielectric tube and are arranged in a length direction of the outer dielectric tube and fluid-communicate with the second space, wherein a source gas is injected through the plurality of through-holes into the outer dielectric tube, wherein the in-water plasma discharge means generates plasma using the source gas such that the generated plasma is directed from the outer dielectric tube to the treatment target water flowing through the second space.

In one implementation of the system, the in-water plasma discharge means includes a plurality of in-water plasma discharge means received in the second space and arranged along a flow direction of the treatment target water.

A second aspect of the present disclosure provides a spray nozzle for spraying plasma-discharge treated water as droplets, the spray nozzle comprising: a rotatable drum including a circular bottom and a circular sidewall in a form of a cylinder, wherein the sidewall extends from the bottom in a perpendicular direction to the bottom, wherein a plurality of water spray slits extend radially, and are defined in a circular top of the sidewall, and are arranged along the circular top of the sidewall; a drum rotation shaft connected to the bottom of the rotatable drum and configured to rotate so as to rotate the rotatable drum; and a fluid supply configured to supply fluid to an inner space of the rotatable drum, wherein fluid injected into the inner space of the rotatable drum flows along an inner face of the sidewall toward the top of the sidewall under a centrifugal force according to rotation of the rotatable drum, and then, the fluid reaching the top of the sidewall is atomized into droplets through the spray slits, and then the droplets are ejected toward a space around the rotatable drum.

In one implementation of the spray nozzle, the rotatable drum includes an inner drum and an outer drum, wherein the inner drum include a first circular bottom and a first circular sidewall in a form of a cylinder, wherein the sidewall extends from the bottom in a perpendicular direction to the bottom, wherein the outer drum includes a second circular bottom and a second circular sidewall in a form of a cylinder, wherein the second sidewall extends from the bottom in a perpendicular direction to the second bottom, wherein an outer face of the inner drum and an inner face of the outer drum are spaced from each other by a predefined spacing to define a fluid guide space therebetween, wherein the plurality of water spray slits are defined in a circular top of the second sidewall of the outer drum, and are arranged along the circular top of the second sidewall, wherein the fluid supply is constructed to supply the fluid to the fluid guide space.

In one implementation of the spray nozzle, the predefined spacing is sized such that the fluid flows along and in the fluid guide space in a form of a thin film.

In one implementation of the spray nozzle, a vertical level of a top of the first sidewall of the inner drum is higher than a vertical level of the top of the second sidewall of the outer drum.

In one implementation of the spray nozzle, each of the water spray slits is tapered such that a width thereof decreases as the slit extends radially outwardly.

In one implementation of the spray nozzle, the sidewall extends upwardly in an inclined manner at an obtuse angle relative to the bottom.

In one implementation of the spray nozzle, the sidewall includes: an inclined portion extending upwardly in an inclined manner at an obtuse angle relative to the bottom; and a vertical extension wall extending from a distal end of the inclined wall along a direction perpendicular to the bottom, wherein the water spray slits are defined in a circular top of the vertical extension wall.

In one implementation of the spray nozzle, the spray nozzle further comprises a water receiving member installed so as to be coaxial with the rotatable drum, wherein the water receiving member surrounds the bottom and the sidewall of the rotatable drum, wherein the water receiving member is embodied as a container having an open top of a larger diameter than a diameter of the rotatable drum, and is constructed to receive the fluid overflowing out of an upper end of the rotatable drum.

According to the disinfection system using the plasma-discharge treated water according to the present disclosure, the plasma-discharge treated water may be atomized into the droplets which in turn may be sprayed in an aerosol form, such that the plasma-discharge treated water may be suspended in the air for a longer time, and thus may be efficiently used to sterilize existing bacteria and viruses.

### BRIEF DESCRIPTION OF DRAWINGS

FIG. 1 is a diagram schematically showing a configuration of a disinfection system using plasma-discharge treated water according to one embodiment of the present disclosure.

FIG. 2 is a view showing a first embodiment of a plasma-discharge treated water generation device according to the present disclosure.

FIG. 3 is a diagram for illustrating a process in which plasma discharge occurs in in-water plasma discharge means of the plasma-discharge treated water generation device according to the first embodiment.

FIG. 4 is a diagram showing a second embodiment of the plasma-discharge treated water generation device according to the present disclosure.

FIG. 5 is a diagram showing a third embodiment of the plasma-discharge treated water generation device according to the present disclosure.

FIG. 6 is an enlarged cross-sectional view of the in-water plasma discharge means shown in FIG. 5.

FIG. 7 is a view showing a first embodiment of a spray nozzle according to the present disclosure.

FIG. 8 is a view showing a second embodiment of the spray nozzle according to the present disclosure.

FIG. 9 is a view showing a third embodiment of the spray nozzle according to the present disclosure.

FIG. 10 is a view showing a fourth embodiment of the spray nozzle according to the present disclosure.

FIG. 11 is a view showing a fifth embodiment of the spray nozzle according to the present disclosure.

### DETAILED DESCRIPTION OF THE INVENTION

Hereinafter, with reference to the accompanying drawings, a disinfection system using plasma-discharge treated water according to an embodiment of the present disclosure and a spray nozzle for spraying plasma-discharge treated water as droplets will be described in detail. An embodiment of the present disclosure will be described in detail with reference to the accompanying drawings. The present disclosure may be variously modified and may take many forms. Thus, specific embodiments will be illustrated in the drawings and described in detail herein. However, the specific embodiments are not intended to limit the present disclosure thereto. It should be understood that all changes, equivalents thereto, or substitutes therewith are included in a scope and spirit of the present disclosure. In describing the drawing, similar reference numerals are used for similar components. A shape, a size, a ratio, an angle, a number, etc. disclosed in the drawings for illustrating embodiments of the present disclosure are illustrative, and the present disclosure is not limited thereto.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the idea and scope of the present disclosure.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or greater other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

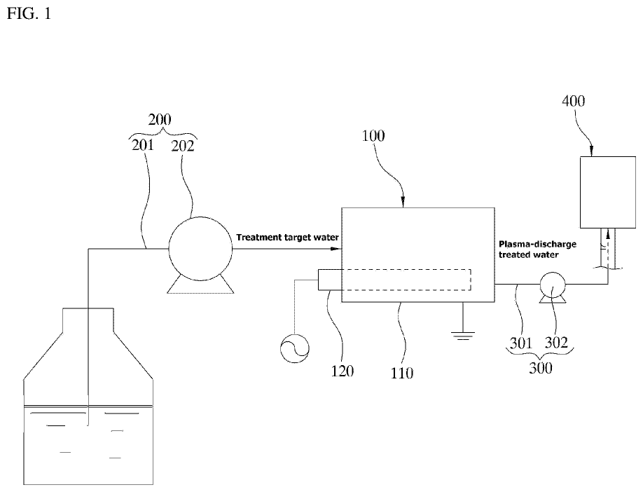

FIG. 1 is a diagram schematically showing a configuration of a disinfection system using plasma-discharge treated water according to one embodiment of the present disclosure.

Referring to FIG. 1, the disinfection system using plasma-discharge treated water according to one embodiment of the present disclosure includes a plasma-discharge treated water generation device 100, a treatment target water supply 200 for supplying treatment target water to the plasma-discharge treated water generation device 100, a plasma-discharge treated water discharger 300 for discharging the plasma-discharge treated water from the plasma-discharge treated water generation device 100 to an outside, and a spray nozzle 400 for spraying the plasma-discharge treated water.

The plasma-discharge treated water generation device 100 is configured to generate plasma in the treatment target water supplied from the treatment target water supply 200 to treat the treatment target water with plasma to generate plasma-discharge treated water.

The treatment target water supply 200 supplies the treatment target water to the plasma-discharge treated water generation device 100. In one example, the treatment target water supply 200 may be configured to pump the treatment target water from a storage space (not shown) where the treatment target water is stored and supply the pumped treatment target water to the plasma-discharge treated water generation device 100. Although not shown, for example, the treatment target water supply 200 may include a treatment target water supply pipe 201 connecting the plasma-discharge treated water generation device 100 and the storage space where the treatment target water is stored to each other, and a first pump 202 installed on the treatment target water supply pipe 201.

The plasma-discharge treated water discharger 300 may be connected to one side of the plasma-discharge treated water generation device 100 and may discharge the plasma-discharge treated water generated from the plasma-discharge treated water generation device 100 to a component out of the plasma-discharge treated water generation device 100. In one example, the plasma-discharge treated water discharger 300 may include a plasma-discharge treated water discharge pipe 301 connected between the plasma-discharge treated water generation device 100 and the spray nozzle 400, and a second pump 302 installed on the plasma-discharge treated water discharge pipe 301.

The spray nozzle 400 is connected in fluid communication with the plasma-discharge treated water generation device 100 so that plasma-discharge treated water is supplied from the plasma-discharge treated water discharger 300 to the spray nozzle 400. The spray nozzle 400 may atomize the supplied plasma-discharge treated water into droplets and spray the droplets.

[symbol]

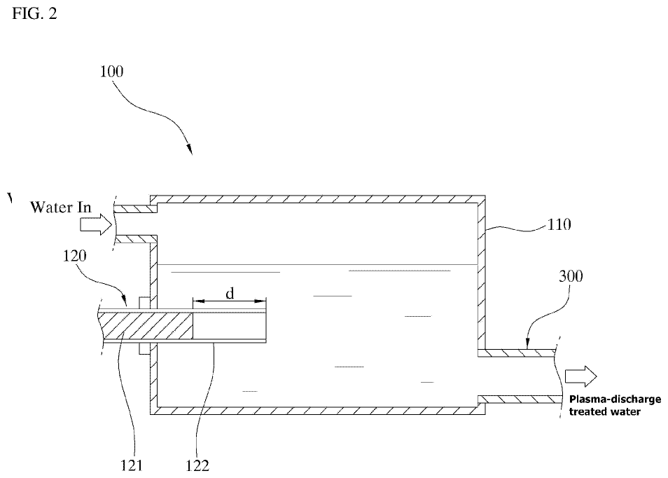

FIG. 2 is a view showing a first embodiment of the plasma-discharge treated water generation device according to the present disclosure.

Referring to FIG. 2, the plasma-discharge treated water generation device 120 includes a chamber 110 and an in-water plasma discharge means 120. The chamber 110 is connected to the treatment target water supply 200 and thus receives the treatment target water supplied from the treatment target water supply 200. The in-water plasma discharge means 120 is partially received in an inner space of the chamber 110 so as to generate plasma in the treatment target water.

The in-water plasma discharge means 120 may include a metal tip 121 to which power is applied, and a dielectric tube 122 that surrounds the metal tip 121 and protrudes by a predefined length d beyond a distal end of the metal tip 121. In this regard, d may be appropriately determined in consideration of micro-sized vapor phase bubbles produced inside the dielectric tube 122 and discharge effect generated in the micro-sized vapor phase bubble. The dielectric tube 122 may be made of, for example, quartz.

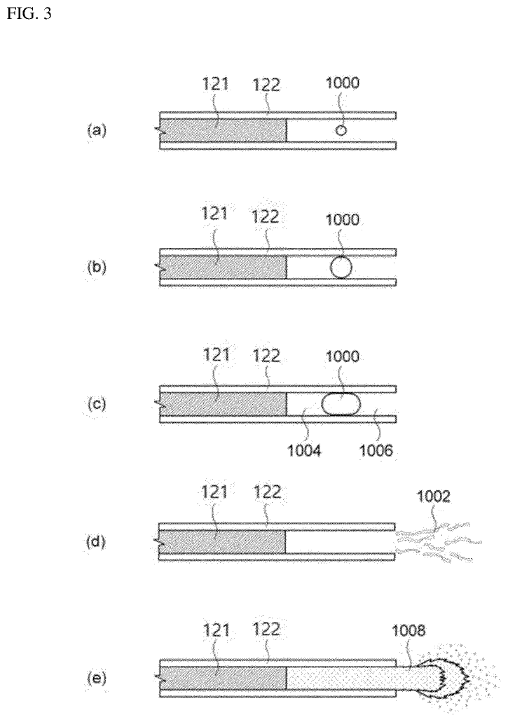

FIG. 3 is a diagram for illustrating a process in which plasma discharge occurs in the in-water plasma discharge means of the plasma-discharge treated water generation device according to the first embodiment.

First, it is assumed that a voltage supplied to the metal tip 121 is Vp. When |Vp| reaches about 150V, the micro-sized vapor phase bubble 1000 is produced inside the dielectric tube 122 as shown in (a) of FIG. 3. A main component of the micro-sized vapor phase bubble is hydrogen generated under electrolysis. Thereafter, as |Vp| increases, a size of the micro-sized vapor phase bubble 1000 increases due to Joule heating, and eventually becomes equal to an inner diameter of the dielectric tube 122 (See (b) in FIG. 3).

When |Vp| reaches about 1180V, an intensity of Joule heat generated by surface discharge inside the dielectric tube 122 becomes stronger by restricted current inside the dielectric tube 122 such that the micro-sized vapor phase bubble 1000 is pushed toward an inlet of the dielectric tube 122, and a shape of the micro-sized vapor phase bubble 1000 changes from a circular shape to an elliptical shape (See (c) in FIG. 3). Further, when the shape of the micro-sized vapor phase bubble 1000 becomes elliptical, a contact area between the micro-sized vapor phase bubble 1000 and the dielectric tube 122 becomes larger as shown in FIG. 3. Accordingly, the intensity of the Joule heat applied to the micro-sized vapor phase bubble 1000 is further increased.

Thereafter, when |Vp| continues to increase to about 2680 V, the micro-sized vapor phase bubble 1000 bursts into several vapor phase bubbles 1002. In this regard, a length of the micro-sized vapor phase bubble 1000 is about 4 mm (See (d) in FIG. 3). When the micro-sized vapor phase bubble is completely generated inside the dielectric tube 122, two water columns 1004 and 1006 respectively generated on both opposing sides of the micro-sized vapor phase bubble may act as electrodes such that electrical discharge occurs inside the micro-sized vapor phase bubble. When |Vp| is sufficiently increased 3 to about 6090V, plasma discharge 1008 occurs and is directed out of the dielectric tube 122 as shown in (e) of FIG. (see (e) of FIG. 3).

[symbol]

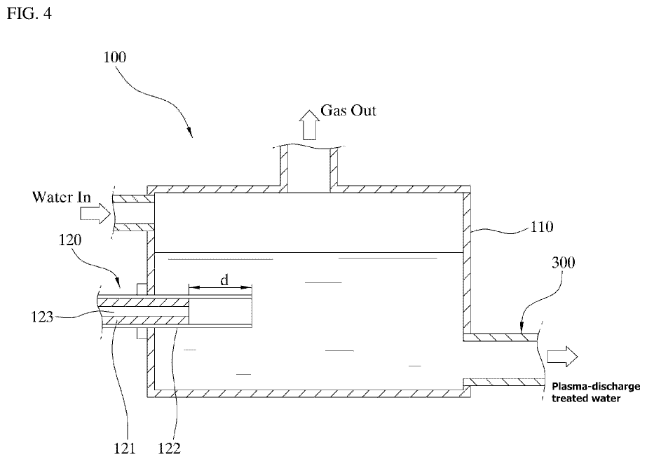

FIG. 4 is a diagram showing a second embodiment of the plasma-discharge treated water generation device according to the present disclosure.

Referring to FIG. 4, the plasma-discharge treated water generation device 100 includes the chamber 110 and the in-water plasma discharge means 120. The chamber 110 is connected to the treatment target water supply 200 and receives the treatment target water supplied from the treatment target water supply 200. The in-water plasma discharge means 120 is partially received in an inner space of the chamber 110 so as to generate plasma in the treatment target water.

The in-water plasma discharge means 120 may have a structure for generating in-water capillary plasma discharge. That is, the in-water plasma discharge means 120 may include the metal tip 121 to which power is applied, the dielectric tube 122 that surrounds the metal tip 121 and protrudes by the predefined length d beyond the distal end of the metal tip 121, and a gas supply pipe 123 extending through the metal tip 121 in a longitudinal direction thereof.

The metal tip 121 may receive the power supplied from a power supply (not shown). The metal tip 121 and the dielectric tube 122 may generate capillary plasma discharge in the treatment target water in the chamber 110 using the applied power. The plasma generated under the capillary plasma discharge decomposes water molecules in the treatment target water to generate active species such as OH⁻, O, H, H₂O₂, HO₂, HClO, Cl₂, and HCl.

The gas supply pipe 123 injects auxiliary gas into the treatment target water where the capillary plasma discharge occurs using the metal tip 121 and the dielectric tube 122. Examples of such auxiliary gas may be ozone (O₃), oxygen (O₂), nitrogen (N₂), argon (Ar), helium (He), air or a mixture thereof. The gas supply pipe 123 may spray liquid hydrogen peroxide (H₂O₂). The auxiliary gas injected in this way is supplied to the plasma generated from the metal tip 121 and the dielectric tube 122, thereby assisting the generation of the plasma. That is, when the auxiliary gas is injected as described above, a concentration of the active species in the treatment target water and a residence time thereof in the treatment target water are increased compared to a case where the auxiliary gas is not injected. Further, when the auxiliary gas is supplied in this way, the plasma may be generated even at a smaller power supply amount than that in the case when the auxiliary gas is not injected.

[symbol]

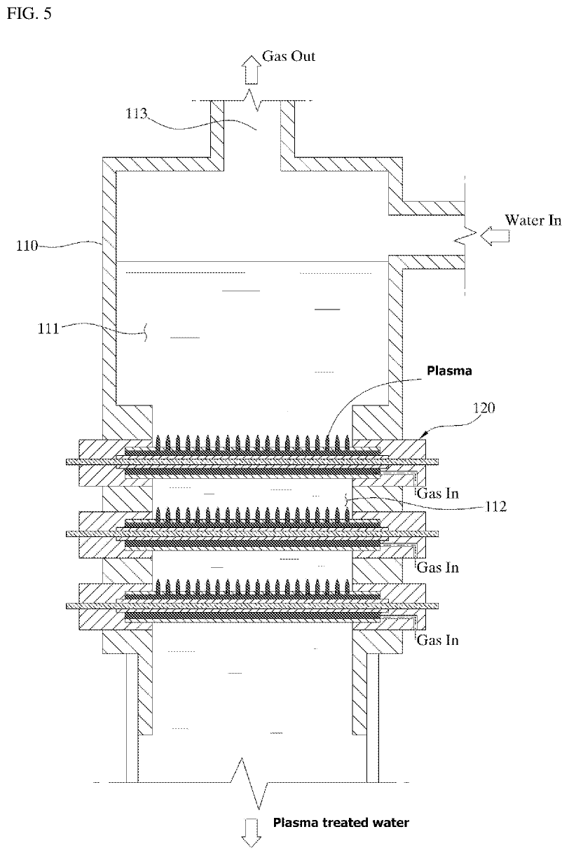

FIG. 5 is a diagram showing a third embodiment of the plasma-discharge treated water generation device according to the present disclosure, and FIG. 6 is an enlarged cross-sectional view of an in-water plasma discharge means as shown in FIG. 5.

Referring to FIG. 5 and FIG. 6, the plasma-discharge treated water generation device includes the chamber 110 and the in-water plasma discharge means 120.

The chamber 110 has a first space 111 defined therein to accommodate the treatment target water therein, and a second space 112 defined therein that is adjacent to and communicates with the first space 111. The plasma-discharge treated water discharger 300 is connected to the second space 112. The chamber 110 has a gas outlet 113 communicatively connected to the first space 111. A gas may be incorporated in the treatment target water supplied to the first space 111 such that the water contains dissolved oxygen.

The in-water plasma discharge means 120 may include a high voltage electrode 121 to which a high voltage is applied, an inner dielectric tube 122 surrounding the high voltage electrode 121, and an outer dielectric tube 123 accommodating the inner dielectric tube 122 therein so that an inner face of the outer dielectric tube 123 is spaced, by a predefined spacing, from an outer face of the inner dielectric tube 122. The in-water plasma discharge means 120 may be installed so that both opposing sides of the outer dielectric tube 123 are inserted into a wall of the chamber 110.

The outer dielectric tube 123 may include a through-hole 123a and a gas supply 123b.

The through-hole 123a may have a hole shape extending through a wall of the outer dielectric tube 123, and the through-holes 123a may be arranged along a longitudinal direction of the outer dielectric tube 123 and may be defined in one side of the wall of the outer dielectric tube 123.

The gas supply 123b is connected to one side of the outer dielectric tube 123, and a source gas may be injected into the outer dielectric tube 123 via the gas supply 123b. The source gas may include carbon dioxide, nitrogen, oxygen, air, an inert gas, or a mixture of at least two thereof.

The high voltage is applied to the high voltage electrode 121 of the in-water plasma discharge means 120, and the chamber 110 or the treatment target water in the chamber 110 may be grounded.

When the power is applied to the high voltage electrode 121, the electrical discharge is generated in a space between the high voltage electrode 121 and the outer dielectric tube 123, so that the source gas is ionized to generate plasma. The plasma passes through the through-hole 123a of the outer dielectric tube 123 and is directed toward the treatment target water. Thus, the plasma decomposes water molecules in the treatment target water to generate active species such as OH⁻, O, H.

Preferably, the source gas may be a mixture of air and oxygen. When air and oxygen are mixed with each other, ozone is generated and the ozone is dissolved in the treatment target water to further enhance the disinfection effect. When OH radicals meet each other, hydrogen peroxide (H₂O₂) is generated, which is more effective in the disinfection. The micro-sized bubble generated under the plasma contains captured ions with high oxidizing power such as O₂⁻, which may be even more effective in the disinfection.

A plurality of in-water plasma discharge means 120 may be arranged along a flow direction of the treatment target water and may be received in the second space 112 of the chamber 110, and may generate the plasma in the treatment target water flowing from the first space 111 to the plasma-discharge treated water discharger 300.

In the plasma-discharge treated water generation device 100 according to this third embodiment, when the treatment target water flows toward the plasma-discharge treated water discharger 300, the water may be treated repeatedly with the plasma generated from the plurality of in-water plasma discharge means 120 while passing by the plurality of in-water plasma discharge means 120. Thus, a larger amount of active species may be contained in the water. Thus, the disinfection effect may be increased.

[symbol]

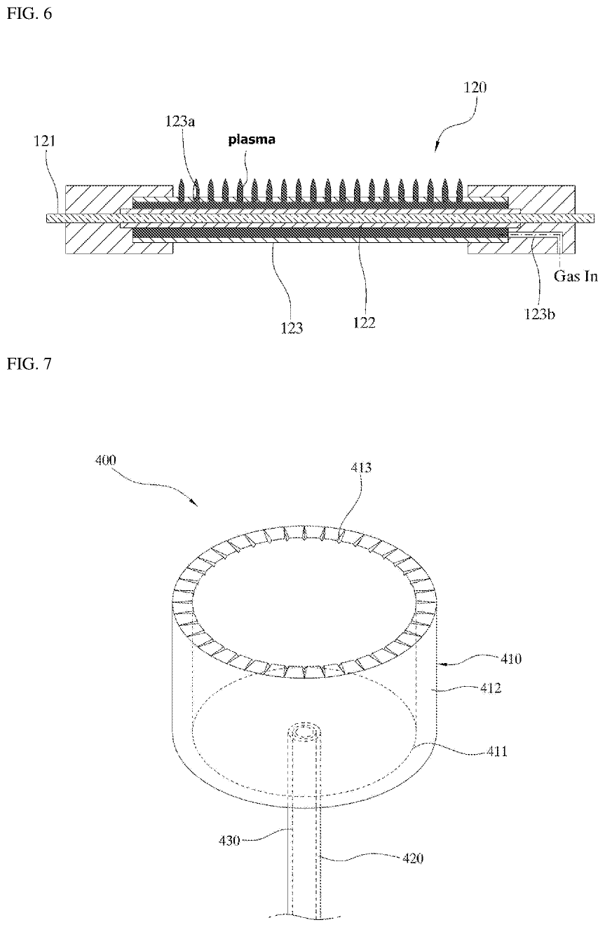

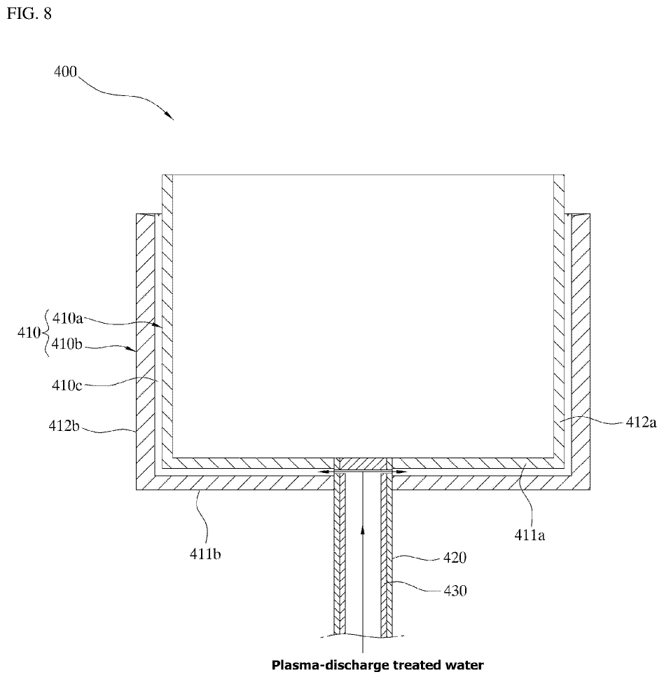

FIG. 7 is a view showing a first embodiment of a spray nozzle according to the present disclosure.

Referring to FIG. 7, the spray nozzle 400 may include a rotatable drum 410, a drum rotation shaft 420 and a fluid supply 430.

The rotatable drum 410 may include a circular bottom 411 and a cylindrical sidewall 412 extending from the bottom 411 in a perpendicular manner to the bottom 411. A plurality of water spray slits 413 extend radially and are defined in a circular top of the sidewall 412 and are arranged along the circular top of the sidewall. The water spray slit 413 is tapered such that a width thereof decreases as the slit extends in a direction from an inner face to an outer face of the sidewall 412.

The drum rotation shaft 420 may be connected to the bottom 411 of the rotatable drum 410 and may rotate so as to rotate the rotatable drum 410. In order for the drum rotation shaft 420 to rotate, the drum rotation shaft 420 may be connected to power means. The power means is not limited to a particular structure. For example, the power means may have a structure in which power is transmitted to the drum rotation shaft 420 via a motor and a belt.

The fluid supply 430 may be configured to supply fluid to an inner space of the rotatable drum 410. For example, the drum rotation shaft 420 may be hollow, and the fluid supply 430 may be provided in a form of a hose or a pipe which may be received in the hollow shaft. One end of the fluid supply 430 is connected to the plasma-discharge treated water discharger 300, and the other end of the fluid supply 430 is inserted into the hollow drum rotation shaft 420 and then extends through the bottom 411 of the rotatable drum 410 so as to supply the fluid into the inner space of the rotatable drum 410.

The spray nozzle 400 may operate as follows. The fluid, that is, the plasma-discharge treated water injected into the inner space of the rotatable drum 410 through the fluid supply 430 may flow along the inner face of the sidewall 412 of the rotatable drum 410 toward the top face thereof under a centrifugal force according to the rotation of the rotatable drum 410. Then, the plasma-discharge treated water reaching the top face of the sidewall 412 may be atomized into droplets while passing through the water spray slits 413 arranged along the top of the sidewall 412. Then, the atomized droplets may be ejected to a space around the rotatable drum 410.

[symbol]

FIG. 8 is a view showing a second embodiment of the spray nozzle according to the present disclosure.

Referring to FIG. 8, the spray nozzle 400 includes the rotatable drum 410, the drum rotation shaft 420 and the fluid supply 430. The rotatable drum 410 includes an inner drum 410a and an outer drum 410b, each having a bottom 411 and a sidewall 412. An outer face of the inner drum 410a and an inner face of the outer drum 410b are spaced from each other by a predefined spacing to define a fluid guide space 410c therebetween. The plurality of water spray slits 413 are defined in a circular top of the sidewall 412 of the outer drum 410b. The fluid supply 430 communicates with the fluid guide space 410c to supply the fluid to the fluid guide space 410c.

For example, the drum rotation shaft 420 may be hollow, and the fluid supply 430 may be provided in a form of a hollow tube, and the fluid supply 430 may be inserted into the hollow drum rotation shaft 420. The fluid supply 430 and the drum rotation shaft 420 may respectively have fluid channels communicating with each other and defined along in a circumferential direction so that the plasma-discharge treated water from the fluid supply 430 is supplied to the fluid guide space 410c through the fluid channels.

Further, the spacing between the inner drum 410a and the outer drum 410b may be sized such that the fluid may flow in a form of a thin film along and in the fluid guide space 410c.

Further, a vertical level of the top of the sidewall 412 of the inner drum 410a is higher than a vertical level of the top of the sidewall 412 of the outer drum 410b. Accordingly, the fluid flowing along the fluid guide space 410c may not overflow the sidewall 412 of the inner drum 410a and may be easily directed toward the top of the sidewall 412 of the outer drum 410b.

In the spray nozzle 400, the plasma-discharge treated water is supplied to the fluid guide space 410c. When the rotatable drum 410 rotates, the plasma-discharge treated water supplied to the fluid guide space 410c flows in a form of the thin film along and in the fluid guide space 410c and flows toward the top of the sidewall 412 of the outer drum 410b under a centrifugal force. When the fluid reaches the top of the sidewall 412 of the outer drum 410b, the fluid may be atomized into droplets while passing through the water spray slits 413 and then the droplet may be ejected to a space around the rotatable drum 410.

In this regard, the plasma-discharge treated water may flow along and in the fluid guide space 410c so that it may be easier to form the fluid in a thin film than it may be when the first embodiment free of the fluid guide space 410c. Loss of the plasma-discharge treated water occurring when the plasma-discharge treated water is scattered within the rotatable drum 410 and thus leaks out of the rotatable drum 410 may be minimized. Thus, a substantial amount of the plasma-discharge treated water fed to the rotatable drum 410 may be used for disinfection.

[symbol]

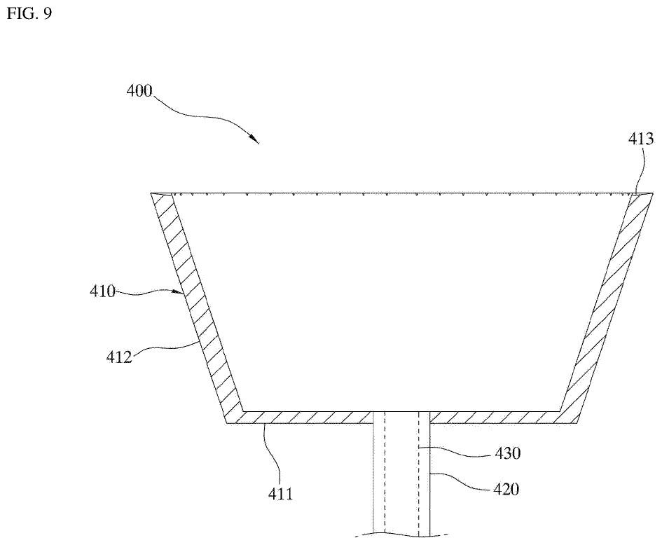

FIG. 9 is a view showing a third embodiment of the spray nozzle according to the present disclosure.

Referring to FIG. 9, the spray nozzle 400 includes the rotatable drum 410, the drum rotation shaft 420 and the fluid supply 430. The rotatable drum 410 includes the bottom 411 and the sidewall 412. The sidewall 412 extends in an upwardly and outwardly inclined manner at an obtuse angle relative to the bottom 411, and a number of water spray slits 413 radially extend and are arranged along the circular top of the sidewall 412 and are defined in the top thereof.

The drum rotation shaft 420 and the fluid supply 430 in this embodiment are respectively the same as the drum rotation shaft 420 and the fluid supply 430 of the spray nozzle 400 of the first embodiment as described above with reference to FIG. 7. Thus, a detailed description thereof will be omitted.

In the spray nozzle 400 of this embodiment in which the sidewall 412 extends in an upwardly and outwardly inclined manner, when the plasma-discharge treated water supplied to the inner space of the rotatable drum 410 flows along the inner face of the sidewall 412 of the rotatable drum 410 toward the top of the sidewall 412 under the centrifugal force generated when the rotatable drum 410 rotates, a greater centrifugal force may act due to the inclined sidewall. Thus, the plasma-discharge treated water may flow quickly and stably to the top of the sidewall 412, and then may more easily enter the multiple water spray slits 413. Accordingly, spraying efficiency of the spray nozzle 400 may be increased.

[symbol]

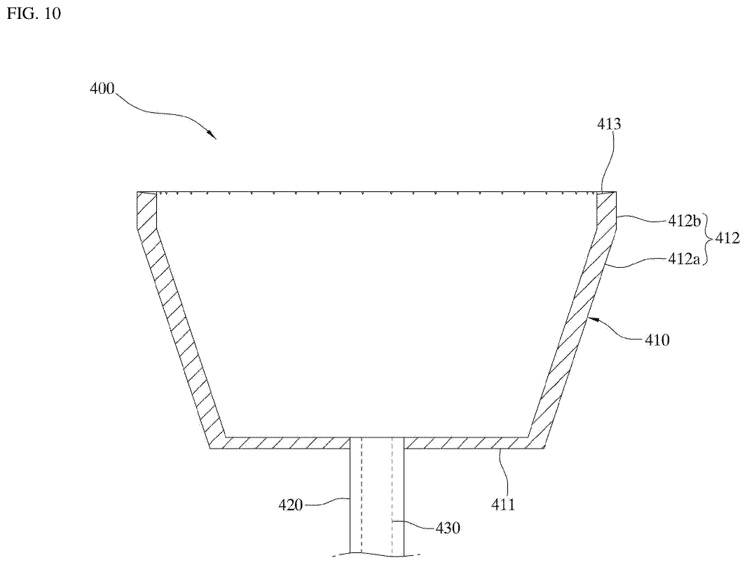

FIG. 10 is a view showing a fourth embodiment of the spray nozzle according to the present disclosure.

Referring to FIG. 10, the spray nozzle 400 includes the rotatable drum 410, the drum rotation shaft 420 and the fluid supply 430. The rotatable drum 410 includes the bottom 411 and the sidewall 412. The sidewall 412 includes an inclined wall 412a extending upwardly at an obtuse angle relative to the bottom 411 and a vertical extension wall 412b extending from a distal end of the inclined wall 412a in a direction perpendicular to the bottom 411. A plurality of water sprays slits 413 may be defined in a circular top of the vertical extension wall 412b and may extend radially and may be arranged along the top thereof.

The drum rotation shaft 420 and the fluid supply 430 in this embodiment are respectively the same as the drum rotation shaft 420 and the fluid supply 430 of the spray nozzle 400 of the first embodiment as described above with reference to FIG. 7. Thus, a detailed description thereof will be omitted.

In the spray nozzle 400 of this embodiment, the plasma-discharge treated water supplied to the inner space of the rotatable drum 410 flows along the inner face of the inclined wall 412a of the sidewall 412 of the rotatable drum 410 to a top of the inclined wall 412a, and reaches a boundary of the inclined wall 412a and the vertical extension wall 412b, and then flows along an inner face of the vertical extension wall 412b to the top of the vertical extension wall 412b. At this time, since the inclined wall 412a of the sidewall 412 is inclined, the centrifugal force generated when the rotatable drum 410 rotates may be greater such that the fluid flows quickly and stably to the top of the vertical extension wall 412b, and more easily enters the multiple water spray slit 413. Accordingly, the spraying efficiency of the spray nozzle 400 may be increased.

[symbol]

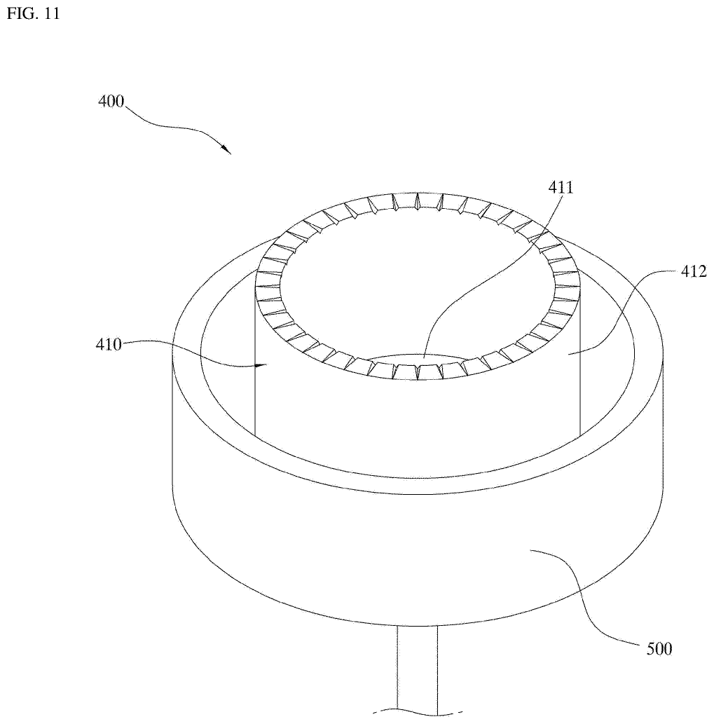

FIG. 11 is a view showing a fifth embodiment of the spray nozzle according to the present disclosure.

Referring to FIG. 11, the spray nozzle 400 of the fifth embodiment is identical with the spray nozzle 400 according to the first embodiment as described with reference to FIG. 7, except that the spray nozzle of the fifth embodiment further includes a water receiving member 500. A following description is mainly about the water receiving member 500.

The water receiving member 500 is installed so as to coaxial with the rotatable drum 410 so that the member 500 may surround the bottom 411 and the sidewall 412 of the rotatable drum 410. This water receiving member 500 is embodied as a container with an open top of a diameter larger than that of the rotatable drum 410, so that the fluid leaking out of an upper end of the rotatable drum 410 may be accommodated in the member 500.

[symbol]

Hereinafter, a process from the supply of the treatment target water to the spraying of the plasma-discharge treated water in the disinfection system according to one embodiment of the present disclosure will be described.

First, the treatment target water is supplied from the treatment target water supply 200 to the plasma-discharge treated water generation device 100.

The treatment target water supplied from the treatment target water supply 200 is received in the chamber 110 of the plasma-discharge treated water generation device 100. The in-water plasma discharge means 120 is located in the treatment target water accommodated in the chamber 110. The in-water plasma discharge means 120 generates plasma in the treatment target water. Thus, the treatment target water is plasma-treated, and thus, the active species are generated in the treatment target water. The plasma-discharge treated water is discharged out of the plasma-discharge treated water generation device 100 through the plasma-discharge treated water discharger 300.

The plasma-discharge treated water discharged through the plasma-discharge treated water discharger 300 is supplied to the spray nozzle 400. In this regard, the plasma-discharge treated water is supplied to the inner space of the rotatable drum 410 through the fluid supply 430 of the spray nozzle 400, and the drum rotation shaft 420 is rotated so as to rotate the rotatable drum 410.

When the rotatable drum 410 rotates, the treated water contained in the rotatable drum 410 flows along the inner face of the sidewall 412 upwardly in a thin film that adheres to the sidewall 412 of the rotatable drum 410 under the centrifugal force caused by the rotation of the rotatable drum 410, and then reaches the top of the sidewall 412. The plasma-discharge treated water reaching the top of the sidewall 412 flows into the plurality of water spray slits 413 defined in the top of the sidewall 412.

At this time, the treated water flowing into the water spray slits 413 is split from the thin film into droplets and flows into the water spray slits 413. The droplet flows along the water spray slit 413 whose the width gradually decreases as the slit extends radially outwardly. Thus, a size of the droplet gradually decreases as the droplet flows along the water spray slit 413. At a distal end of the water spray slit 413, the droplet is atomized into a micro-sized droplet that in turn may be sprayed in the air around the rotatable drum 410.

Thus, according to the disinfection system using the plasma treated water according to the first embodiment of the present disclosure, the treatment target water may be plasma-treated to generate the plasma-discharge treated water. The plasma-discharge treated water may be atomized into the droplets which in turn may be sprayed in an aerosol form, such that the plasma-discharge treated water may be suspended in the air for a longer time, and thus may be efficiently used to sterilize bacteria and viruses present in the air.

Further, the use of the plasma-discharge treated water is not limited to the disinfection. The plasma treated water may be sprayed on a surface of an object to be sterilized, and thus may be used for sterilization of the object.

Further, the spray nozzle 400 atomizes the plasma-discharge treated water into droplets and sprays the droplets in an aerosol form, so that the plasma-discharge treated water may be easily used for the disinfection.

Although not shown, the disinfection system using the plasma-discharge treated water according to the present disclosure may be configured in a portable form and may be displaced to a contaminated area and disinfect the area or may be configured so as to be installed on the ground for disinfection of vehicles, etc.

The descriptions of the presented embodiments have been provided so that a person of ordinary skill in the art of any the present disclosure may use or practice the present disclosure. Various modifications to these embodiments will be apparent to those skilled in the art of the present disclosure, and the general principles defined herein may be applied to other embodiments without departing from the scope of the present disclosure. Thus, the present disclosure is not intended to be limited to the embodiments presented herein, but is to be construed in the widest scope consistent with the principles and novel features presented herein.

1. A disinfection system using plasma-discharge treated water, the system comprising:
a plasma-discharge treated water generation device to generate the plasma-discharge treated water;
a treatment target water supply for supplying treatment target water to the plasma-discharge treated water generation device;
a plasma-discharge treated water discharger connected to one side of the plasma-discharge treated water generation device; and
a spray nozzle connected to the plasma-discharge treated water generation device in a fluid communication manner,
wherein the spray nozzle is constructed to:
receive the plasma-discharge treated water from the plasma-discharge treated water discharger; and
atomize the received plasma-discharge treated water into droplets and spray the droplets.

2. The system of claim 1, wherein the plasma-discharge treated water generation device includes:
a chamber receiving therein the treatment target water; and
in-water plasma discharge means received in an inner space of the chamber so as to generate plasma in the treatment target water.

3. The system of claim 2, wherein the in-water plasma discharge means includes:
a metal tip receiving an electrical power; and
a dielectric tube surrounding the metal tip and protruding by a predefined length beyond a distal end of the metal tip.

4. The system of claim 2, wherein the in-water plasma discharge means includes:
a metal tip receiving an electrical power;
a dielectric tube surrounding the metal tip and protruding by a predefined length beyond a distal end of the metal tip; and
a gas supply channel extending through the metal tip in a longitudinal direction of the tip.

5. The system of claim 2, wherein the in-water plasma discharge means includes:
a high voltage electrode to which a high voltage is applied;
an inner dielectric tube surrounding the high voltage electrode; and
an outer dielectric tube constructed to accommodate therein the inner dielectric tube so that an inner face of the outer dielectric tube is spaced, by a predefined spacing, from an outer face of the inner dielectric tube,
wherein a plurality of through-holes are defined in a wall of the outer dielectric tube and are arranged in a length direction of the outer dielectric tube,
wherein a source gas is injected through the plurality of through-holes into the outer dielectric tube,
wherein the in-water plasma discharge means generates plasma using the source gas such that the generated plasma is directed from the outer dielectric tube toward the treatment target water.

6. The system of claim 1, wherein the plasma-discharge treated water generation device includes:
a chamber having:
first and second inner spaces defined therein and adjacent to each other and connected to each other in a fluid communicate manner, wherein the treatment target water is received in the first space, and the plasma-discharge treated water discharger is connected to the second space; and
a gas outlet connected to the first inner space in a fluid communicate manner; and
in-water plasma discharge means received in the second space so as to generate plasma in the treatment target water flowing from the first space toward the plasma-discharge treated water discharger,
wherein the in-water plasma discharge means includes:
a high voltage electrode to which a high voltage is applied;
an inner dielectric tube surrounding the high voltage electrode; and
an outer dielectric tube constructed to accommodate therein the inner dielectric tube so that an inner face of the outer dielectric tube is spaced, by a predefined spacing, from an outer face of the inner dielectric tube,
wherein a plurality of through-holes are defined in a wall of the outer dielectric tube and are arranged in a length direction of the outer dielectric tube and fluid-communicate with the second space,
wherein a source gas is injected through the plurality of through-holes into the outer dielectric tube,
wherein the in-water plasma discharge means generates plasma using the source gas such that the generated plasma is directed from the outer dielectric tube to the treatment target water flowing through the second space.

7. The system of claim 6, wherein the in-water plasma discharge means includes a plurality of in-water plasma discharge means received in the second space and arranged along a flow direction of the treatment target water.

8. A spray nozzle for spraying plasma-discharge treated water as droplets, the spray nozzle comprising:
a rotatable drum including a circular bottom and a circular sidewall in a form of a cylinder, wherein the sidewall extends from the bottom in a perpendicular direction to the bottom, wherein a plurality of water spray slits extend radially, and are defined in a circular top of the sidewall, and are arranged along the circular top of the sidewall;
a drum rotation shaft connected to the bottom of the rotatable drum and configured to rotate so as to rotate the rotatable drum; and
a fluid supply configured to supply fluid to an inner space of the rotatable drum,
wherein fluid injected into the inner space of the rotatable drum flows along an inner face of the sidewall toward the top of the sidewall under a centrifugal force according to rotation of the rotatable drum, and then, the fluid reaching the top of the sidewall is atomized into droplets through the spray slits, and then the droplets are ejected toward a space around the rotatable drum.

9. The spray nozzle of claim 8, wherein the rotatable drum includes an inner drum and an outer drum, wherein the inner drum include a first circular bottom and a first circular sidewall in a form of a cylinder, wherein the sidewall extends from the bottom in a perpendicular direction to the bottom, wherein the outer drum includes a second circular bottom and a second circular sidewall in a form of a cylinder, wherein the second sidewall extends from the bottom in a perpendicular direction to the second bottom,
wherein an outer face of the inner drum and an inner face of the outer drum are spaced from each other by a predefined spacing to define a fluid guide space therebetween,
wherein the plurality of water spray slits are defined in a circular top of the second sidewall of the outer drum, and are arranged along the circular top of the second sidewall,
wherein the fluid supply is constructed to supply the fluid to the fluid guide space.

10. The spray nozzle of claim 9, wherein the predefined spacing is sized such that the fluid flows along and in the fluid guide space in a form of a thin film.

11. The spray nozzle of claim 9, wherein a vertical level of a top of the first sidewall of the inner drum is higher than a vertical level of the top of the second sidewall of the outer drum.

12. The spray nozzle of claim 8, wherein each of the water spray slits is tapered such that a width thereof decreases as the slit extends radially outwardly.

13. The spray nozzle of claim 8, wherein the sidewall extends upwardly in an inclined manner at an obtuse angle relative to the bottom.

14. The spray nozzle of claim 8, wherein the sidewall includes:
an inclined portion extending upwardly in an inclined manner at an obtuse angle relative to the bottom; and
a vertical extension wall extending from a distal end of the inclined wall along a direction perpendicular to the bottom,
wherein the water spray slits are defined in a circular top of the vertical extension wall.

15. The spray nozzle of claim 8, wherein the spray nozzle further comprises a water receiving member installed so as to be coaxial with the rotatable drum, wherein the water receiving member surrounds the bottom and the sidewall of the rotatable drum,
wherein the water receiving member is embodied as a container having an open top of a larger diameter than a diameter of the rotatable drum, and is constructed to receive the fluid overflowing out of an upper end of the rotatable drum.
